(19) United States
(12) Patent Application Publication
(10) Pub. No.: US 2025/0025317 A1
(43) Pub. Date: Jan. 23, 2025

(54) Implant Inserter Having a Laterally-Extending Dovetail Engagement Feature

(71) Applicant: DePuy Synthes Products, Inc., Raynham, MA (US)

(72) Inventors: Thomas Gamache, Westport, MA (US); Michael D. Sorrenti, Middleboro, MA (US)

(21) Appl. No.: 18/906,581

(22) Filed: Oct. 4, 2024

Related U.S. Application Data
(63) Continuation of application No. 17/806,984, filed on Jun. 15, 2022, now Pat. No. 12,109,127.
Application No. 17/806,984 is a continuation of application No. 16/517,837, filed on Jul. 22, 2019, now Pat. No. 11,382,768.
Application No. 16/517,837 is a continuation-in-part of application No. 13/364,280, filed on Feb. 1, 2012, now Pat. No. 10,369,015.
Application No. 13/364,280 is a continuation-in-part of application No. 13/237,174, filed on Sep. 20, 2011, now Pat. No. 11,529,241.
Application No. 13/364,280 is a continuation-in-part of application No. 13/237,200, filed on Sep. 20, 2011, now abandoned.
Application No. 13/364,280 is a continuation-in-part of application No. 13/237,233, filed on Sep. 20, 2011, now abandoned.
(60) Provisional application No. 61/466,309, filed on Mar. 22, 2011.
(60) Provisional application No. 61/466,321, filed on Mar. 22, 2011.
(60) Provisional application No. 61/385,959, filed on Sep. 23, 2010.
(60) Provisional application No. 61/466,309, filed on Mar. 22, 2011.
(60) Provisional application No. 61/466,321, filed on Mar. 22, 2011.
(60) Provisional application No. 61/385,959, filed on Sep. 23, 2010.
(60) Provisional application No. 61/466,309, filed on Mar. 22, 2011.
(60) Provisional application No. 61/466,321, filed on Mar. 22, 2011.
(60) Provisional application No. 61/385,959, filed on Sep. 23, 2010.

(51) Int. Cl. A61F 2/46; A61B 17/02; A61B 17/16; A61B 17/17; A61B 17/80; A61B 17/88; A61F 2/30; A61F 2/44

(52) U.S. Cl. CPC A61F 2/4611; A61B 17/1631; A61B 17/808; A61B 17/8875; A61F 2/4455; A61F 2/447; A61B 2017/0256; A61B 17/1757; A61B 17/80; A61F 2002/30387; A61F 2002/30507; A61F 2002/30578; A61F 2002/30593; A61F 2002/30823; A61F 2002/30904; A61F 2002/4627; A61F 2002/4629; A61F 2002/4635; A61F 2002/4687

(57) ABSTRACT

A method of inserting a fastener into a fusion cage can include attaching a fastener head of a bone fastener to a flexible bone fastener driver. The method can also include inserting the bone fastener into a threaded throughhole of a fusion cage comprising a front wall, a pair of opposing side walls, a back wall, and top and bottom surfaces adapted for gripping opposed vertebral endplates, wherein the front wall comprises the threaded throughhole.

### CROSS-REFERENCE TO RELATED APPLICATIONS

This is a continuation of U.S. Ser. No. 17/806,984 filed Jun. 15, 2022, which is a continuation of U.S. Ser. No. 16/517,837 filed Jul. 22, 2019, which is a continuation-in-part of U.S. Ser. No. 13/364,280, filed Feb. 1, 2012, which is a continuation-in part of U.S. Ser. No. 13/237,174 filed Sep. 20, 2011, which claims priority to U.S. Ser. No. 61/466,309, filed on Mar. 22, 2011, U.S. Ser. No. 61/466,321 filed Mar. 22, 2011, and U.S. Ser. No. 61/385,959 filed Sep. 23, 2010. U.S. Ser. No. 13/364,280 is also a continuation-in-part of U.S. Ser. No. 13/237,200 filed Sep. 20, 2011, which claims priority to U.S. Ser. No. 61/466,309 filed Mar. 22, 2011, U.S. Ser. No. 61/466,321 filed Mar. 22, 2011, and U.S. Ser. No. 61/385,959 filed Sep. 23, 2010. U.S. Ser. No. 13/364,280 is also a continuation-in-part of Ser. No. 13/237,233 filed Sep. 20, 2011, which claims priority to U.S. Ser. No. 61/466,309 filed Mar. 22, 2011, U.S. Ser. No. 61/466,321 filed Mar. 22, 2011, and U.S. Ser. No. 61/385,959 filed Sep. 23, 2010, the specifications of which are incorporated by reference in their entireties.

### BACKGROUND OF THE INVENTION

Spine surgeons have expressed a desire to locate an implant with a low vertical profile instrument to minimize retraction and increase visibility of the implant. Ideally, the instrument and its connection point are sized to be fully contained within the profile of the implant, thus being smaller than the implant and allowing easy positioning or placement of the implant. Additionally, the connection to the instrument should be desirably rigid, with no toggle or rotation in any plane. This rigidity prevents implant movement during the passing of instruments or bone screw insertion.

Conventional inserter instruments possessing chamfer features typically work in one plane, wherein a grabber tip collapses upon the implant in a medial/lateral direction and thereby secures the implant to the instrument. However, even with exacting tolerances, toggle often appears if the user of the conventional instrument were to apply a moment to the posterior edge of the implant (in flexion/extension) in one plane. Conventional inserters with chamfer features also do not take advantage of dissimilar angles at the interface in more than one plane, and so fail to ensure that the anterior surface of the implant bottoms out on the inserter grabber tip.

U.S. Pat. No. 5,443,514 (Steffee) discloses an inserter that grips the side of a spinal implant. Sec FIGS. 4-6 of Steffee.

US Patent Publication No. 2005-0143749 (Zalenski) discloses an inserter having engagement features forming a vertically-extending dovetail shape. Sec FIGS. 2A-2C of Zalenski.

### SUMMARY OF THE INVENTION

The present invention relates to an intervertebral implant having a novel “compound angle” recess, such as a dovetail shape, designed and sized to couple to a delivery instrument. The interface between the implant and securing instrument is designed with laterally-extending, angled chamfer features extending in more than one direction to prevent toggle and rotation. Once the split tip instrument is collapsed to its closed position, the implant is drawn into the inserter and allowed to bottom out on a distal wall of the inserter.

The instrument of the present invention is advantageous over conventional inserter instruments having threads and other known grabber features, which sometimes need to withstand impaction and may move upon insertion of a bone anchor or instruments through the device.

The dual angle nature of the present invention ensures that the implant is axially square and/or co-linear with the instrument and so further ensures that a larger surface area of the instrument absorbs any required impaction.

The present invention may also have an added angle, wherein the dimension on the anterior portion of the feature is sized to be smaller than the posterior portion of the feature. See FIG. 6.

In particular embodiments, the instrument of the present invention has a forked inner shaft with distal tynes extending therefrom, wherein each distal tyne forms a medially-extending dovetail feature. This dovetail feature possesses the compound angulation that prevents the undesired movements discussed above.

In preferred embodiments of the present invention, the proximal wall of the implant has a corresponding pair of recesses opening onto its respective lateral walls. Each of these recesses forms a dovetail silhouette on its respective lateral wall of the implant.

The invention is generally related to a method and apparatus for assisting in a safe, one-handed insertion of an implant. The implant implantation device generally includes (i) a frame that includes a trigger mechanism, (ii) an outer sleeve mechanically coupled to the frame, (iii) a forked inner shaft having distal tynes for mechanically engaging an implant, the forked inner shaft slidably disposed within the outer sleeve and (iv) a retaining element for directing the distal tynes towards a closed position. The retaining element can be a spring.

Optional elements on the inserter may include a knob, a drag adjustment screw, at least one protrusion, and a depth control member. The knob can be mechanically coupled to the outer sleeve for causing the outer sleeve and the forked inner shaft to be rotated about the frame. The drag adjustment screw can provide tension between the trigger mechanism and the forked inner shaft. The at least one protrusion can be located on the outer sleeve for slidably engaging a distraction instrument. The depth control member can be slidably coupled to the outer sleeve for providing a predetermined insertion depth of the implant.

The distal tynes of the inserter hold the implant therebetween during insertion of the implant between the vertebrae. Each distal tyne includes an engagement feature at its tip for mechanically engaging the implant. The engagement feature comprises a dovetail-shaped protrusion extending in the lateral direction. Generally, the engagement feature has a medial face, with each medial face having a proximal end portion having a height and a distal end portion having a height, wherein the height of the distal end portion is greater than the height of the proximal end portion. At the same time, the front wall of the implant comprises a pair of mating laterally-extending dovetail recesses opening onto a respective side wall and forming a dovetail silhouette on each side wall, wherein each engagement feature of the instrument is received in a respective recess of the spinal implant.

The forked inner shaft can include at least one marking to identify a position of the implant in relation to the patient. The marking can be a pin located on a surface of the forked inner shaft. The marking can be a plurality of machined slots on a surface of the forked inner shaft.

Mechanically engaging the implantation instrument to the implant may include the steps of (i) opening the forked inner shaft located on an end of the implantation instrument, (ii) aligning the tynes of the forked inner shaft with the recesses of the implant, and (iii) closing the tynes to mechanically engage the tynes to the implant.

The method may further include the steps of (iv) distracting a prepared disc space with a distraction instrument, (v) inserting the implant into the prepared disc space with the implantation instrument, (vi) releasing the implant from the implantation instrument, and (vii) removing the implantation instrument and distraction instrument.

In some embodiments, inserting the implant into a prepared disc space may include the step of aligning the implantation instrument with the distraction instrument.

The invention has many advantages. For example, the invention provides safe one-handed insertion of an implant into a prepared disc space. The invention reduces the amount of time required to complete the surgical procedure. The invention also provides for various manipulations of the implant without physically contacting the implant. For example, the invention can align an endplate of the implant radially and provide a lordotic angle for implantation. The invention can be used for packaging the implant, and the invention can be used to hold the implant during the implant sterilization process.

Therefore, in accordance with the present invention there is provided an instrument for inserting an implant, comprising;
a) an outer sleeve having a bore, and
b) a forked member having a proximal rod and a pair of distal tynes extending therefrom, each distal tyne comprising a proximal portion and a distal portion having a distal wall having an engagement member extending distally therefrom, each engagement member having a medial face forming a dovetail shape, wherein the proximal rod and the proximal portion of each tyne are slidably received within the bore of the outer sleeve, and wherein the distal portion of each tyne extends out of the bore of the outer sleeve.

Also in accordance with the present invention there is provided an assembly comprising:
a) the above-described inserter, and
b) a spinal implant having a front wall, a pair of opposing side walls and a back wall, wherein the front wall comprises a pair of recesses opening onto a respective side wall to form a dovetail shape in each respective side wall,
wherein each engagement feature of the instrument is received in a respective recess of the spinal implant. Also in accordance with the present invention there is provided an orthopedic implant having a front wall, a pair of opposing side walls, a back wall, and top and bottom surfaces, wherein the front wall comprises a pair of recesses, each recess opening onto a respective side wall and forming a dovetail shape in the respective side wall.

### DESCRIPTION OF THE DRAWINGS

FIG. 1A shows a cross-sectional view of an insertion instrument of the present invention.

FIG. 1B shows a plan view of the insertion instrument of FIG. 1A.

FIGS. 2A-2C show a perspective view of three embodiments of a forked inner shaft of the present invention.

FIG. 3 shows a perspective view of the implant being inserted into a prepared disc space using the insertion instrument of FIGS. 1A-2C.

FIGS. 4A and 4B show two views of an instrument of the present invention having dovetail gripping features approaching an implant having corresponding dovetail recess features.

FIG. 5 discloses an intervertebral fusion cage of the present invention.

FIG. 6 discloses a corner of an implant having a sidewall having recess therein, wherein the recess comprises two acute angles α and β.

FIG. 7 discloses a perspective view of an inserter tip of the present invention.

FIG. 8 discloses a perspective view of a lighted inserter tip of the present invention.

FIGS. 8A-8F disclose a flexible screw inserter and a prebent sleeve.

### DETAILED DESCRIPTION OF THE INVENTION

The foregoing and other objects, features and advantages of the invention will be apparent from the following more particular description of preferred embodiments of the invention, as illustrated in the accompanying drawings in which like reference characters refer to the same parts throughout the different views. The same number appearing in different drawings represents the same item. The drawings are not necessarily to scale, with emphasis instead being placed upon illustrating the principles of the invention.

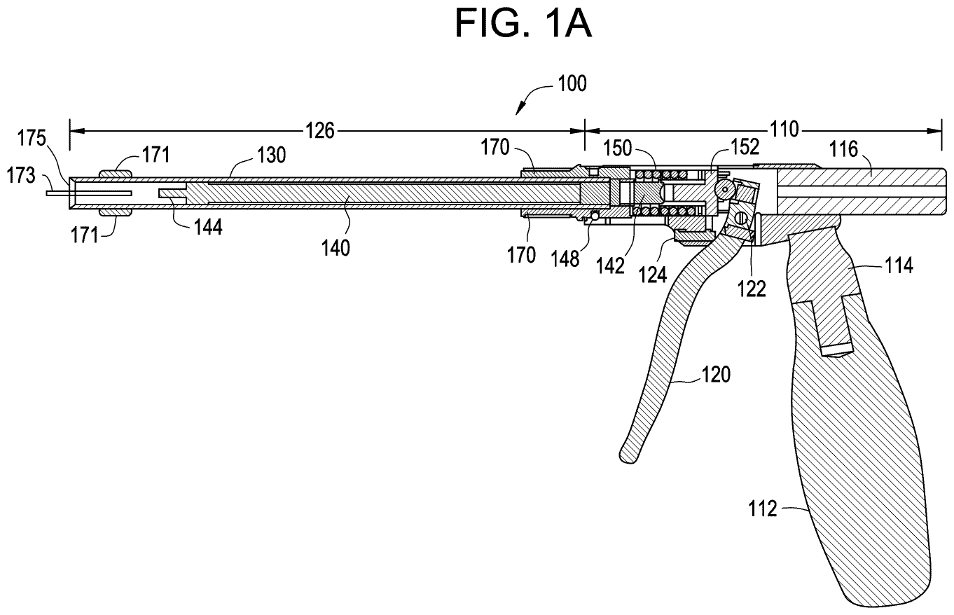

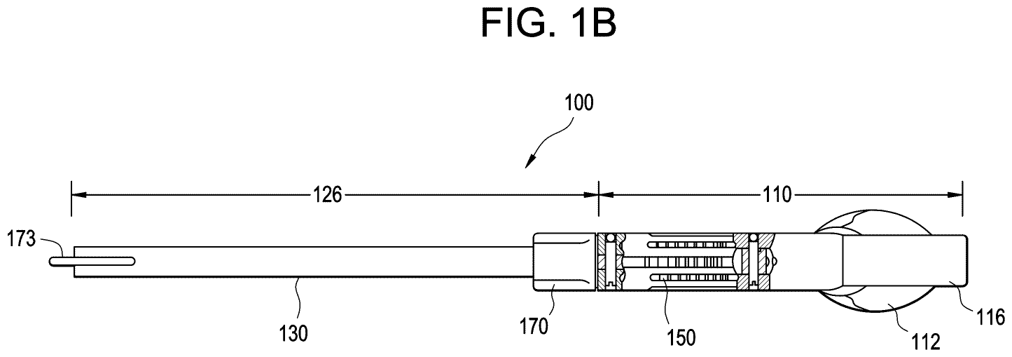

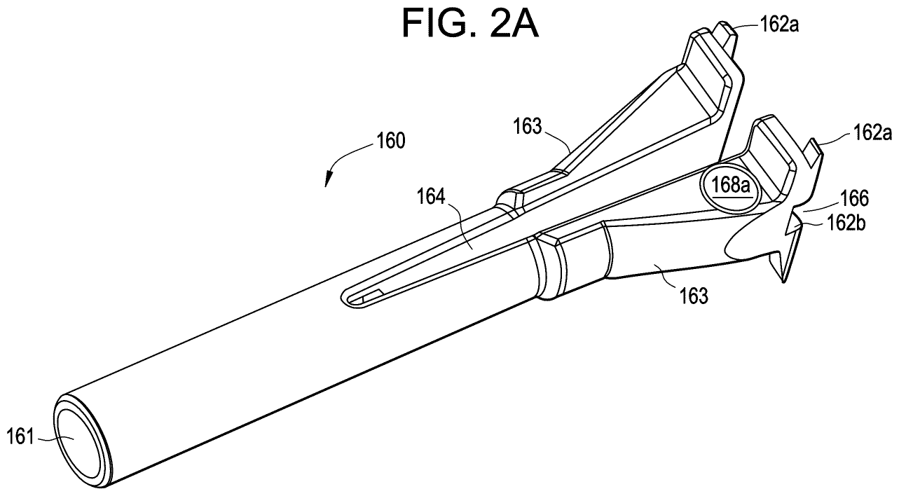

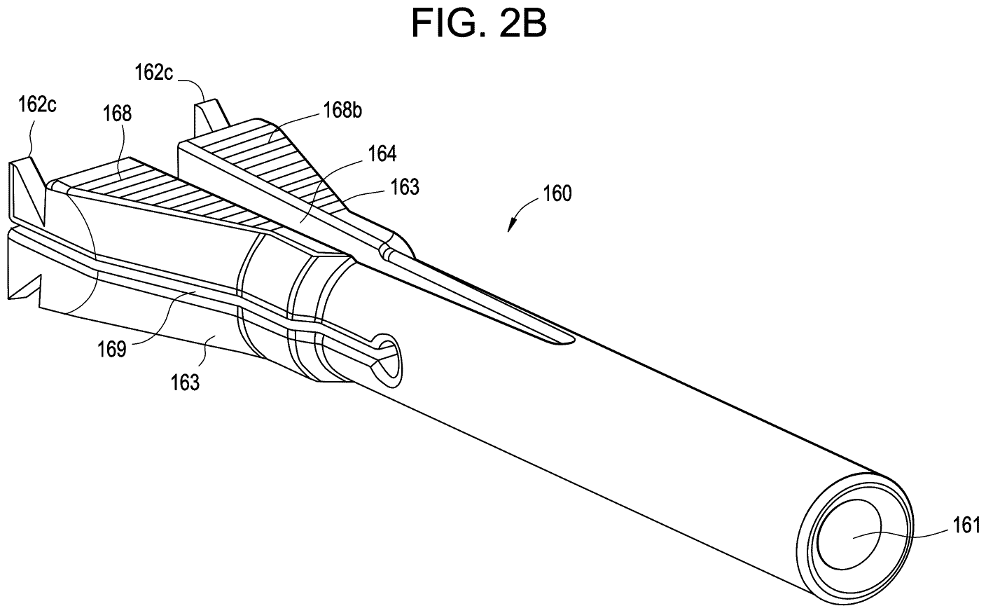

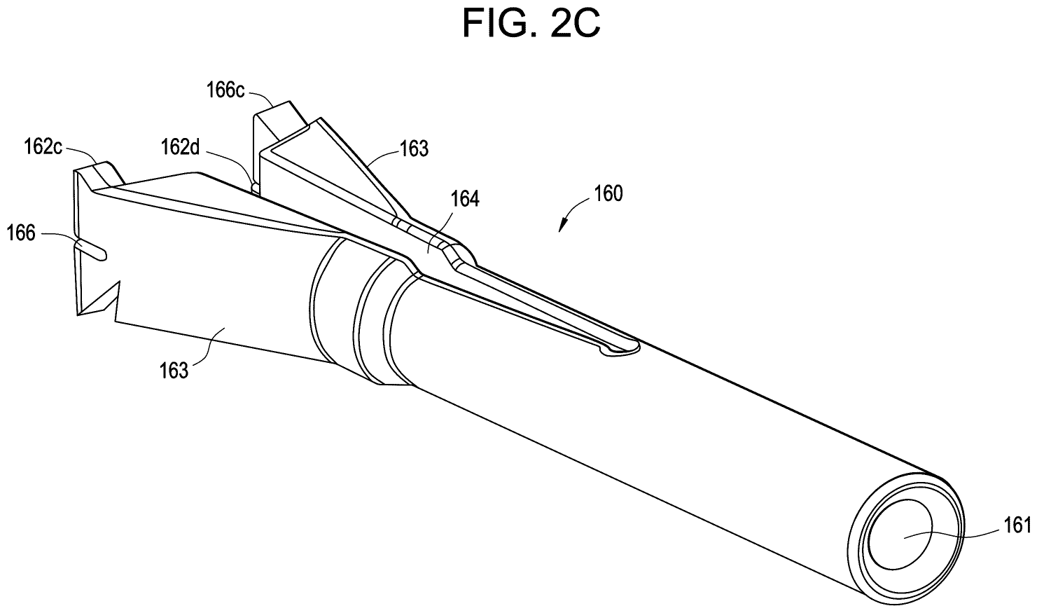

In general, the present invention is related to an apparatus and a method for safely inserting an implant into a spine. The implant can be an artificial disc or spinal fusion cage, or a spinal plate. Referring to FIGS. 1A and 1B, insertion instrument 100 is shown in a side cross-sectional view and a plan view, respectively. Insertion instrument 100 includes a frame or driver body assembly 110, an actuator assembly 126 and a forked inner shaft 160 (FIG. 2A-2C). Insertion instrument 100 is a normally closed device, that is, the proximal rod of the forked inner shaft 160 is normally substantially contained within actuator assembly 126.

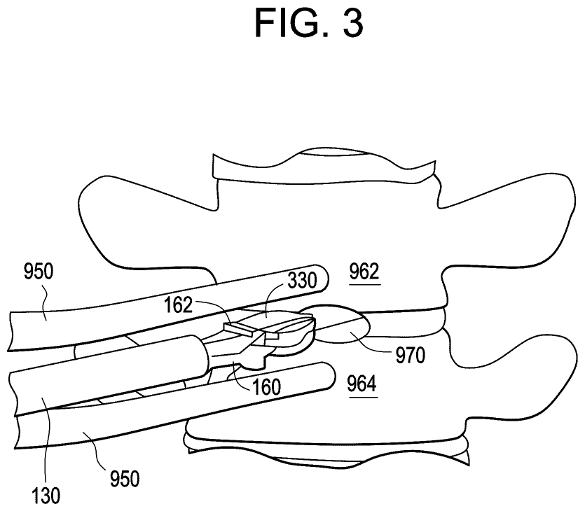

Actuator assembly 126 includes an outer sleeve 130, a proximal inner shaft 140, and a retaining pin 148. Outer sleeve 130 includes a tapered end 175 which slidably engages tapers 163 on the forked inner shaft 160 (FIG. 2A-2C), allowing for compression and expansion of the forked inner shaft 160 when in use. Inner shaft 140 includes a female threaded end 142 and a male threaded end 144. Female threaded end 142 mates with a spring retaining screw 152 and male threaded end 144 mates with the forked inner shaft 160. Internal compression spring 150 is fastened to the actuator assembly 126 and is held in place by spring retaining screw 152. Once actuator assembly 126 is assembled, it is inserted into driver body assembly 110 and retained within assembly 110 with retaining pin 148. Optional knob 170 can be mechanically attached to outer sleeve 130 to allow outer sleeve 130 and proximal inner shaft 140 to rotate about the driver body assembly 110. Optional guides 171 can be attached to outer sleeve 130 to slidably mate with spinal disc distraction instrument 950 (FIG. 3). Depth control member 173 can also be fixedly or slidably attached on outer sleeve 130 for providing a predetermined insertion depth of the implant.

Driver body assembly 110 includes handle 112, handle transition 114, strike boss 116, trigger mechanism 120, and pivot pin 122. Trigger mechanism 120 can be any type of trigger mechanism known in the art. Trigger mechanism 120 pivots about pivot pin 122 in the driver body assembly 110. When trigger mechanism 120 is squeezed toward handle 112, the forked inner shaft 160 (FIG. 2A-2C) extends from actuator assembly 126 and expands to release an implant. When trigger mechanism 120 is released, forked inner shaft 160 recedes into actuator assembly 126 and compresses, thereby engaging the implant or returning to its normally closed position. Optional drag adjustment screw 124 is rotatably coupled to driver body assembly 110 for adjusting the drag force between trigger mechanism 120 and spring retaining screw 152 of actuator assembly 126.

FIGS. 2A-2C show various forked inner shafts 160 of the present invention. Each forked inner shaft 160 includes engagement features 162 for mechanically engaging the implant. engagement features 162 may be various shapes and sizes depending upon implant selection. As shown, engagement features 162 may be dovetail-shaped (162a, 162b, 162c, 162d). Engagement features 162 can engage implants having multiple heights. It should be understood engagement features 162 can be any shape which can engage any type of implant. In an alternative embodiment, proximal inner shaft 140 and forked inner shaft 160 can be integral.

Each forked inner shaft 160 includes female threaded hole 161 for mating to male threaded end 144 of proximal inner shaft 140 of actuator assembly 126. It should be understood that any means known in the art can be used to attach forked inner shaft 160 to proximal inner shaft 140.

Each forked inner shaft 160 includes tapers 163 and relatively long expansion/compression slot 164 to allow forked inner shaft 160 to expand and compress during use. FIGS. 2A-2C show forked inner shaft 160 in the expanded position. Each forked inner shaft 160 also includes sizing slot 166 to allow for a variation of tab and forked inner shaft slot dimensional differences. Expansion/compression slot 169 (FIG. 2B) is an alternative embodiment of sizing slot 166. In some embodiments, the forked member has a quick connect feature.

Cephalad markers 168 can be included on a surface of forked inner shaft 160 to allow the user to determine the position of the implant. Markers 168 can be pin 168a or machined slots 168b. In some embodiments, the inserter (squeeze handle) has a quick connect feature.

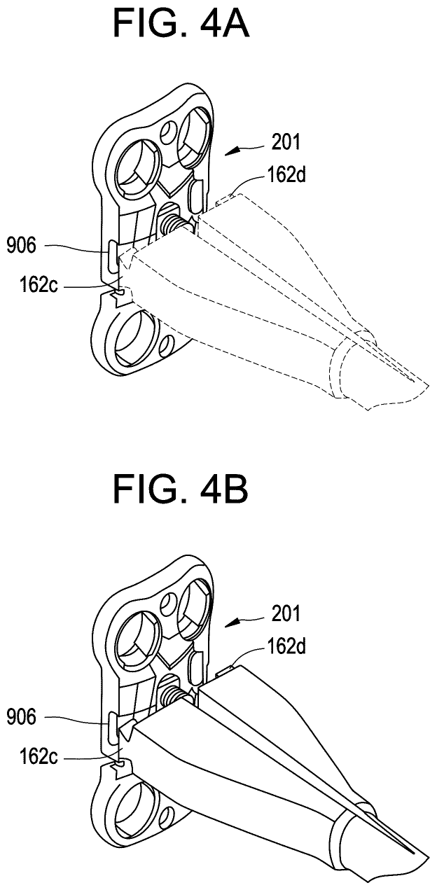

In one method of using the present invention, and now referring to FIGS. 4a and 4b, the user first squeezes the trigger mechanism 120 (FIG. 1) on the implantation instrument 100, thereby causing the engagement features 162c, 162d on the instrument to separate. The user then approaches the target implant (in this case, a cervical plate 201) with the implantation instrument so that the dovetail features of these two devices align, whereby the engagement features 162 straddle the opposed engagement indents 906 on the implant. Once engagement features 162 straddle the engagement indents 906, the user then releases the trigger mechanism 120, causing engagement features 162 to collapse inwards and engage engagement indents 906 on the implant.

In other embodiments, the reverse is provided, wherein the user squeezes the instrument to engage the implant.

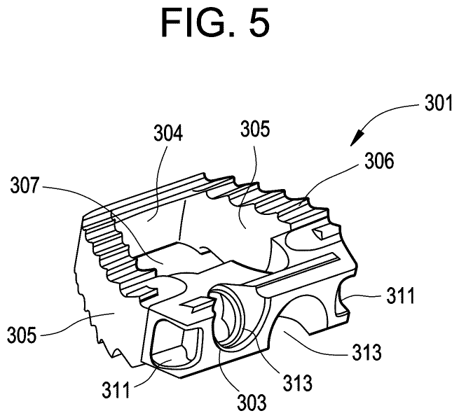

Now referring to FIG. 5, there is provided an intervertebral fusion cage 301 of the present invention. This cage comprises a front wall 303, a back wall 304, a pair of opposing side walls 305 connecting the front and back walls, a top surface 306 adapted to engage an upper vertebra, a bottom surface (not shown) adapted to engage a lower vertebra, and a throughhole 307 extending between the top and bottom surfaces for promoting fusion therethrough, wherein the front wall comprises a pair of recesses 311, each recess opening onto a respective sidewall and forming a dovetail shape in the respective sidewall. The front wall of this particular cage also has screwholes 313 extending therethrough. The screwholes are typically threaded and are adapted to receive bone screws for securing the cage to opposing vertebral bodies without the need for posterior instrumentation, thereby providing “stand alone’ capabilities.

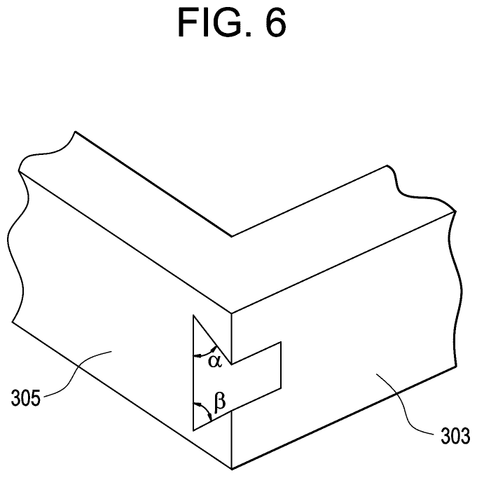

Now referring to FIG. 6, also in accordance with the present invention, there is provided a spinal implant having a front wall, a pair of opposing side walls, a back wall, and top and bottom surfaces, wherein the front wall comprises a pair of recesses, each recess opening onto a respective side wall and forming at least two acute angles α and β in the respective side wall. The acute nature of these two angles in the same recess creates the condition necessary to preclude toggle and rotation.

In some embodiments, the top and bottom surfaces of the implant are spaced at a distance suitable for contacting opposing vertebral endplates. This feature is advantageous for spinal implants inserted into a disc space.

In some embodiments, the implant of the present invention has a throughhole extending from its top surface to its bottom surface. This throughhole feature promotes bony fusion through the implant and so provides a performance advantage for fusion cage embodiments of the present invention.

In some embodiments thereof, a bone graft material is contained in the throughhole of the fusion cage. This graft material also promotes fusion through the implant and so provides a performance advantage for fusion cage embodiments of the present invention.

In some implant embodiments, the fusion cage has a front wall that is manufactured separately from the remainder of the implant. Preferably, the front wall is metallic and the remainder of the implant is polymeric. This preferred embodiment advantageously provides strength in the front wall so that screw holes can pass therethrough without fracturing the wall.

In some embodiments, first and second bone fasteners extend through the screwholes located in the front wall of the fusion cage. This feature allows the cage to be secured to the opposing vertebrae and so eliminates the need for posterior instrumentation. In some embodiments thereof, the first fastener further extends through the top surface of the implant, while the second fastener further extends through the bottom surface of the implant.

In some embodiments, the first and second bone fasteners extend only through the front wall, as in a plate embodiment.

In some embodiments, the implant has an articulation interface, as in an articulating motion disc.

FIG. 3 discloses an instrument of the present invention inserting an implant of the present invention into a disc space.

As shown in FIG. 3, distraction instrument 950 is inserted over pins (not shown) that are secured into vertebral bodies 962, 964. Cervical fusion cage 330 is passed between the forks of distraction instrument 950 using implantation instrument 100 (FIGS. 1A-1B). In an alternate embodiment, guides 170 on insertion instrument 100 slidably engage slots in the forks of distraction instrument 950 to help the user guide cervical fusion cage 330 into prepared disc space 970. Once the cervical fusion cage 330 is in a desired location within prepared disc space 970, the user actuates (e.g., squeezes, or releases or rotates a knob) trigger mechanism 120 (FIG. 1A), which releases cervical fusion cage 330 into the prepared disc space 970. The user can determine the desired position by observing cephalad markers 168 (FIG. 3) located on a surface of forked inner shaft 160. In an alternative embodiment, implantation instrument 100 can include a depth control member 173 (FIG. 1A) (which might be an adjustable and slidable), such that cervical fusion cage 330 can be inserted into prepared disc space 970 at a predetermined depth.

Lastly, the implantation instrument 100 and distraction instrument 950 are removed, causing superior vertebra 962 and inferior vertebra 964 to engage cervical fusion cage 330.

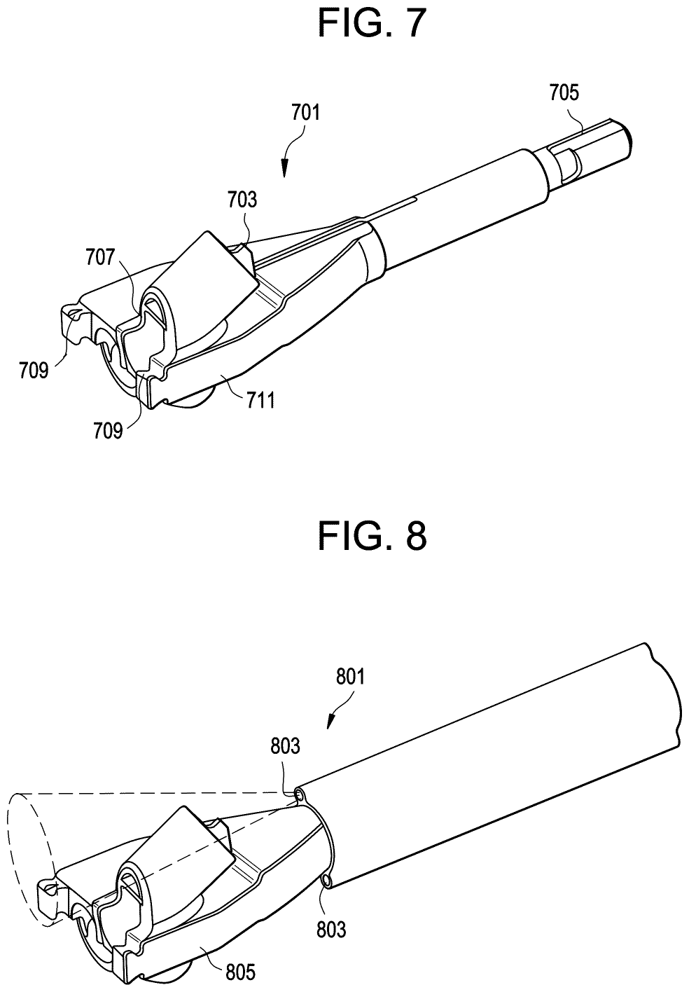

Now referring to FIG. 7, there is provided one embodiment of an inserter tip 701 of the present invention. This inserter tip includes barrel stop 703 that prevents the bone screw from stripping. There is a distal quick connect/release feature 705 that allows for case of connection to the remainder of the inserter instrument. Barrels 707 prevent the implant from being placed too far posteriorly. Relief features 709 on the dovetail allow for the engagement feature to disengage with ease from the implant. Lastly, barrels 711 can be a fixed barrel or adjustable via sliding member such as a ratcheting feature.

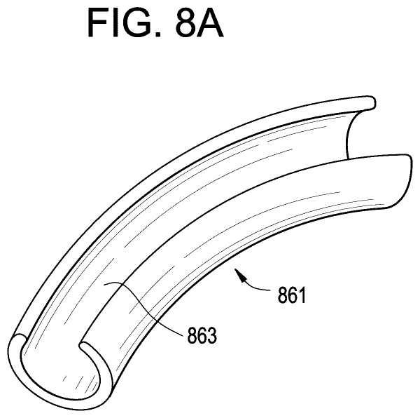

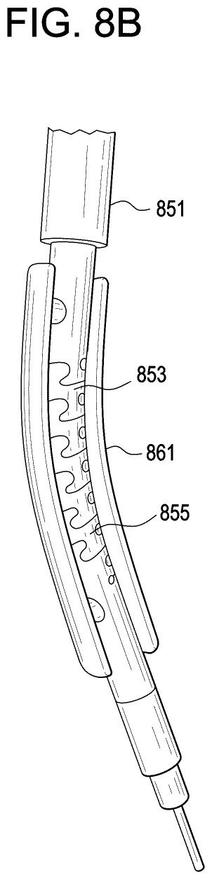

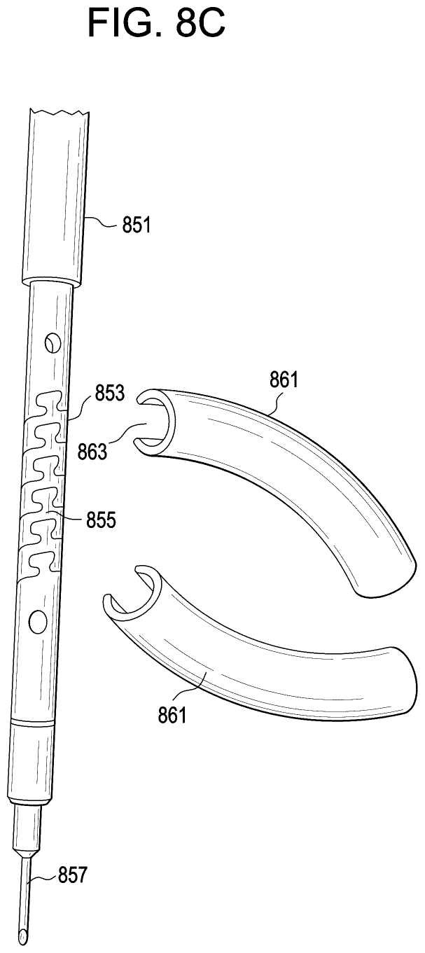

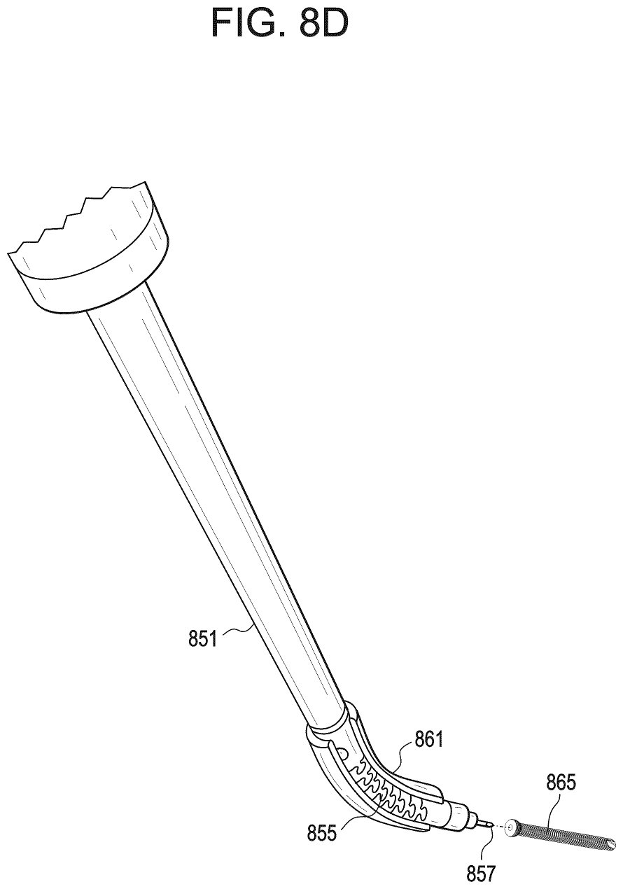

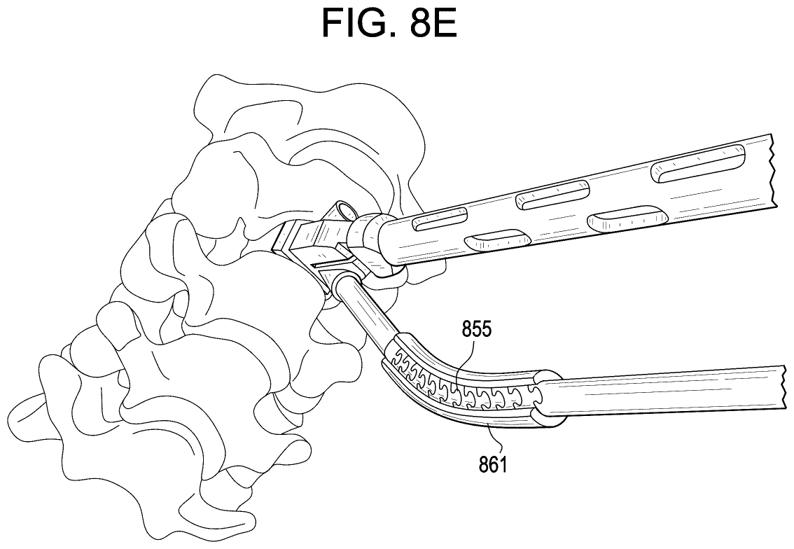

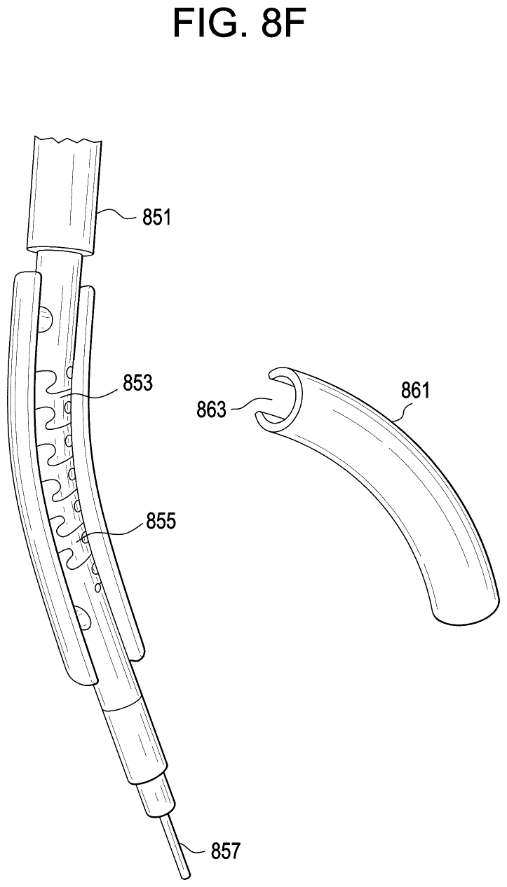

Now referring to FIG. 8, there is provided one embodiment of a lighted inserter tip 801 of the present invention. The inserter cannula is fitted with at least one secondary lumen 803 adapted for delivering light or delivering a fiber optic. Light emanating from the secondary lumen is shown by the dashed lines in FIG. 8. Alternatively, light can be fed down the major lumen that holds the inserter tip (not shown). In one embodiment, at least the engagement features 805 of the tip are made of hard clear plastic that allows visibility of the other instruments passed through the barrels.

When performing a spinal procedure such as an anterior cervical discectomy and fusion (ACDF), the surgeon often has a number of implant options that may achieve the desired clinical outcomes of disc height maintenance and pain relief. Because of their clinical benefits, zero-profile fusion cages that accept bone anchors and are secured to the adjacent anatomy have experienced an increased usage and adoption in the ACDF procedure. These cages typically have angled bone anchors (such as screws) that pass through a portion of the cage's anterior wall and into the adjacent vertebral endplates.

However, if a surgeon were to choose a low profile, stand-alone fusion device (such as a fixation cage comprising one or more bone screws), there remains a surgical technique challenge associated with the insertion of the screws. Traditionally, an instrument having a straight shaft is used to insert the screws through the cage at the steep angle to ensure bone penetration. However, screw insertion with a straight-shaft instrument at locations up near a patient's chin or sternum produces some challenging approach angles. Recently, there have been improvements to the insertion instrument that help facilitate screw placement at these difficult approach angles. Some of these improvements include the adoption of universal joints and flexible inserter instruments that allow for torque transmission while still positioning their handles off-axis from the trajectory of the screw. For example, some flexible inserter instruments achieve flexibility by possessing a plurality of interlocking segments just proximal of their distal working tip. Once the tip of such a flexible driver is inserted into the drill guide, the handle of the driver can be repositioned to the desired location and torque can be transmitted to advance the screw.

However, one of the challenges associated with this flexible technology is that the flexible shaft is typically straight in its resting configuration. Accordingly, one of two procedures needs to be carried out. In a first procedure, the amount of soft tissue retraction has to temporarily be increased in order to engage the tip of the driver into the drill guide and once engaged, the soft tissue retraction can be minimized again and the handle can be held in a desirable position, such as near parallel to the implant inserter. In a second procedure, the tip can be inserted into the drill guide at a less steep angle than the screw trajectory (but not perfectly parallel to the inserter) and the spring force of the flex segment has to be overcome in order for the tip to find the drill guide trajectory. Upon this “turning the corner” step, as a downforce is provided, the self-retention of the screw may become disengaged.

Therefore, to overcome these issues, in one aspect of the present invention, and now referring to FIGS. 8A-8F, there is provided an improvement on the flexible shaft technology that has benefits for the patient and the surgeon. The improvement is a bone screw driver having a) a proximal handle (not shown), b) an intermediate shaft 851, c) a flexible distal end portion 853 comprising a plurality of interlocking segments 855 and distal tip 857 (wherein the flexible distal end portion is substantially straight in its unloaded configuration), and d) a pre-bent sleeve 861 that is placed over and around the flexible segments, thereby pre-determining the trajectory of the tip and facilitating the appropriate and desired insertion angle of the screw into the drill guide without increasing the amount of retraction. As shown in FIGS. 8a-8f, this pre-bent sleeve (which has a longitudinal slit 863) allows a screwdriver handle to be perfectly in-line with the cage inserter if desired, while still taking advantage of the tactile feel and torque transmission of the flex segment in its bent position. Accordingly, a surgeon can limit the amount of retraction and keep the driver handle close to the inserter without having to overcome the forces of the flexible segments' natural straight configuration. The sleeve can be fixed, slidable, or removable and can fully or partially encompass the flexible segments. The sleeve can have geometries that allow for flexing, attachment (snapping-on) to the driver, and removal for cleaning, if desired. The sleeve can be made of any biocompatible material that will maintain the desired shape (such as metal or plastic).

Therefore, in accordance with the present invention, there is provided a flexible bone screw driver comprising:
a) a proximal handle,
b) an intermediate shaft,
c) a flexible distal end portion comprising a plurality of interlocking segments and a distal tip adapted to engage a screw head, and
d) a pre-bent sleeve placed over and around the plurality of interlocking segments.

Also in accordance with the present invention, there is provided an assembly comprising:
a) a fusion cage comprising a front wall, a pair of opposing side walls, a back wall, and top and bottom surfaces adapted for gripping opposed vertebral endplates, wherein the front wall comprises at least one threaded throughhole,
b) a bone screw received in the threaded throughhole, the bone screw having a threaded shaft and a proximal head,
c) the inserter of FIGS. 8A-8F, wherein the distal tip of the inserter is received in the proximal head of the bone screw.

While this invention has been particularly shown and described with references to preferred embodiments thereof, it will be understood by those skilled in the art that various changes in form and details may be made therein without departing from the scope of the invention encompassed by the appended claims.

We claim:

1. An assembly comprising:
a fusion cage including a front wall, a pair of opposing side walls, a back wall, and top and bottom surfaces adapted for gripping opposed vertebral endplates, wherein the front wall comprises a threaded throughhole; and
a flexible bone fastener driver configured to be attached to a fastener head of a bone fastener which is configured to be inserted into the threaded throughhole of the fusion cage.

2. The assembly of claim 1, wherein the flexible bone fastener driver has a proximal handle that is configured, and the flexible bone fastener driver is configured to guide the bone fastener into the threaded throughhole.

3. The assembly of claim 2, wherein the fastener head is configured to be attached to a distal end portion of the bone screw driver, wherein the bone screw driver includes an intermediate shaft that extends from the proximal handle to the distal end portion, and the distal end is bendable.

4. The assembly of claim 3, wherein the distal end comprises a plurality of interlocking segments that are configured to be manipulated so as to bend the distal end portion.

5. The assembly of claim 4, further comprising a pre-bent sleeve that is configured to be coupled to the distal end portion so as to transition the distal end portion from a straight configuration to a curved configuration.

6. The assembly of claim 5, wherein the distal end portion is configured to be transitioned from the straight configuration to the curved configuration prior to inserting the fusion cage at an implant site.

7. The assembly of claim 6, wherein the distal end portion is rotatable while the distal end portion is in the curved configuration such that the bone fastener is configured to threadedly engage a bone.

8. The assembly of claim 6, wherein during rotation of the distal end portion, the handle portion is configured to be maintained substantially parallel to a position of an inserter as the inserter inserts the fusion cage into an intervertebral space.

9. The assembly of claim 7, wherein the pre-bent sleeve is configured to be decoupled from the distal end portion after rotation of the distal end portion.

10. The assembly of claim 5, the pre-bent sleeve is configured to be positioned such that the pre-bent sleeve at least partially envelops the distal end portion so as to couple the pre-bent sleeve to the distal end portion.

11. The assembly of claim 10, wherein the pre-bent sleeve includes a fixed curvature such that the coupling the pre-bend sleeve to the distal end portion causes the distal end portion to adopt the curvature of the pre-bent sleeve.

12. The assembly of claim 5, wherein at least half of an outer periphery of the distal end portion is configured to be enveloped in the pre-bend sleeve so as to couple the pre-bent sleeve to the distal end portion.

13. The assembly of claim 5, wherein the pre-bent sleeve includes a central axis, and the pre-bent sleeve is rotatable about its central axis when a distal tip of the distal end portion is coupled to the fastener head.

14. The assembly of claim 5, the pre-bent sleeve is configured to be coupled to the distal end portion such that a distal end of the pre-bent sleeve is proximal to a distal tip of the distal end portion.

15. The assembly of claim 5, wherein the distal end portion includes a first end and a second end spaced from the first end along a central axis, and the pre-bent sleeve is configured to be slid relative to the distal end along the central axis.

16. The assembly of claim 5, wherein the distal end portion includes a first end and a second end spaced from the first end along a central axis, and a position of the pre-bent sleeve is configured to be fixed relative to the distal end portion along the central axis.

17. The assembly of claim 5, wherein the pre-bent sleeve includes a longitudinal slit, and the distal end portion is configured to be moved through the longitudinal slit so as to coupling the pre-bent sleeve to the distal end portion.

18. The assembly of claim 5, wherein the distal end portion includes a plurality of interlocking segments, and the pre-bent sleeve is configured to be coupled to the plurality of interlocking segments so as to coupling the pre-bent sleeve to the distal end portion.

19. The assembly of claim 2, wherein the distal end portion is flexible.

20. The assembly of claim 5, wherein the pre-bent sleeve is flexible.
